(19) United States
(12) Patent Application Publication
(10) Pub. No.: US 2008/0014284 A1
(43) Pub. Date: Jan. 17, 2008

(54) DISINFECTION OF INSTRUMENTS

(75) Inventors: Bernhard Meyer, Mettmann (DE); Michael Decker, Solingen (DE); Holger Biering, Grevenbroich (DE)

Correspondence Address: ECOLAB INC., MAIL STOP ESC-F7, 655 LONE OAK DRIVE, EAGAN, MN, 55121, US

(73) Assignee: ECOLAB INC., St. Paul, MN (US)

(21) Appl. No.: 11/859,036

(22) Filed: Sep. 21, 2007

Related U.S. Application Data
(63) Continuation of application No. 10/509,806, filed on Oct. 28, 2004.

(30) Foreign Application Priority Data
Apr. 3, 2002 (DE) 101 14 750.7

(51) Int. Cl. A61L 2/23

(52) U.S. Cl. 424/613

(57) ABSTRACT

The invention relates to a powdered disinfectant containing a peroxide, an acylating agent and nonionic surfactants and to the use of this disinfectant for disinfecting surfaces and instruments, particularly in the medical field.

### CROSS REFERENCE TO RELATED APPLICATION

This application is a continuation of U.S. patent application Ser. No. 10/509,806, filed Oct. 28, 2004, the entire disclosure of which is incorporated herein by reference in its entirety.

### FIELD OF THE INVENTION

The present invention relates to a powdered disinfectant containing a peroxide, an acylating agent and nonionic surfactants and to the use of these disinfectants for disinfecting surfaces and instruments, more particularly in the medical field.

### BACKGROUND OF THE INVENTION

Numerous aqueous preparations containing a wide variety of antimicrobial disinfecting ingredients have been proposed over the course of time for the chemical disinfection of instruments. Preparations based on aldehydes have been most widely used in practice, although preparations containing quaternary ammonium compounds, phenols, alcohols and other active disinfecting ingredients are also used. Preparations based on peroxidic active ingredients, more particularly peracetic acid, on the other hand, have rarely been used for this application. This is largely due to the poor stability in storage of these aqueous preparations. Owing to the broad antimicrobial activity of peroxides, attempts have been made to overcome the drawback of poor stability in storage. Thus, it has been proposed, for example in DE-A-26 55 599 and 28 15 400, to prepare the aqueous preparations required for disinfection from more stable precursors, more particularly from sodium perborate and acid anhydrides, just prior to use. According to DE-A-27 01 133, the aqueous preparations are obtained from hydrogen-peroxide-yielding compounds and aromatic acyloxycarboxylic acids. However, only a few of these compounds lead to disinfecting solutions with sufficiently broad activity and these acylating agents mixed with the necessary inorganic peroxides can only be stored for a limited time owing to decomposition reactions. Sekusept powder is the name given to a commercially available product which forms a disinfectant preparation when dissolved in water through the reaction of sodium perborate with tetraacetylethylenediamine (TAED). This product, which is based on an N-acyl compound, has a broad activity spectrum and is stable in storage. Although a high standard of disinfection of medical instruments has been achieved in this way, an improvement in peroxide systems is still being sought to eliminate the remaining gaps in activity and drawbacks in service. A particular drawback of these powdered systems is that they dissolve only very slowly in water. This leads to the disadvantage, on the one hand, that the desired disinfectant concentration is not fully available until a very late stage. On the other hand, there is the additional risk that undissolved constituents will remain in the system to be disinfected or on the surface to be disinfected and will not be rinsed away.

### SUMMARY

It was accordingly an object of the present invention to provide an adequate disinfectant concentration within a short time and thus to minimize the risk of residues in the system and on surfaces.

A further object was to destroy microorganisms, including mycobacteria, in a shorter time.

This object has been achieved by the preparations according to the invention.

### DETAILED DESCRIPTION OF THE INVENTION

Accordingly, the present invention relates to a powdered disinfectant based on active oxygen containing a peracetic acid-generating system consisting of a peroxide and an acylating agent together with nonionic surfactants.

Preferably, the above-mentioned nonionic surfactants are free from alkoxylated alkyl phenols and comprise ether alcohols which are straight-chained or methyl-branched in the 2-position and correspond to the formula:
R—O—(PO)₁₋₂-(EO)₆₋₈-H (I)
where the alkyl and alkenyl radicals R are made up as follows:

C₈=0 to 5% by weight;

C₉₋₁₀=75 to 90% by weight;

C₁₁₋₁₂=5 to 15% by weight;

C₁₃₋₁₄=4 to 10% by weight;

C₁₅₋₁₆=0 to 3% by weight.

The above-mentioned peroxide is preferably selected from the group consisting of sodium perborate monohydrate, sodium perborate tetrahydrate, sodium percarbonate and mixtures thereof.

The above-mentioned acylating agent is preferably selected from the group consisting of tetraacetylglycoluril, tetraacetylethylenediamine, diacetylhexahydrotriazinedione and mixtures thereof.

Other N-acyl compounds which have also been described in detergent chemistry as bleach activators for reactions with hydrogen peroxide in alkaline washing liquors may obviously also be used. Suitable N-acyl compounds are those, in particular, which comprise a further keto group on the acyl group-carrying nitrogen and/or in which the nitrogen is part of a heterocyclic ring system. Examples of suitable N-acyl compounds include the multiply acylated alkylenediamines such as tetraacetylethylenediamine, acylated glycolurils, predominantly tetraacetylglycoluril, N-acylated hydantoins, hydrazides, triazoles, triazines, urazoles, diketopiperazines, sulfurylamides, lactams and cyanurates.

The disinfectant according to the invention preferably contains

10 to 70% by weight, preferably 15 to 60% by weight of the above-mentioned peroxide,

10 to 40% by weight, preferably 15 to 30% by weight of the above-mentioned acylating agent,

0.1 to 10% by weight, preferably 0.5 to 5% by weight, of the above-mentioned nonionic surfactant and

to 100% by weight of soluble inorganic salt and optionally further auxiliaries as remainder.

Further auxiliaries include alkalizing agents, complexing agents for water hardness, complexing agents for heavy metal ions and water-soluble inorganic salts, corrosion inhibitors and other surfactants. The amounts of these auxiliaries in the preparations may vary widely, depending on the intended activity. It does not usually exceed about 3% by weight and is preferably between about 0.001 and about 1% by weight, based on the total preparation.

Sodium triphosphate is mentioned primarily as a complexing agent for the water hardness, though other polyphosphates, salts of nitrilotriacetic acid and salts of organic polycarboxylic acids, for example citric acid, or of polymeric polycarboxylic acids, for example acrylic acid maleic acid copolymers, may also be used for this purpose. Sodium triphosphate, which simultaneously acts as an alkalizing agent, is particularly preferred.

Suitable complexing agents for heavy metal ions which have a decomposing action on peroxide compounds predominantly include aminopolycarboxylic acids and salts thereof, for example ethylenediaminetetraacetic acid, but more particularly aminopolyphosphonic acids such as ethylenediaminetetramethylene phosphonic acid or also hydroxyethane diphosphonic acid and the salts thereof.

Water-soluble salts can act as fillers or builders, such as sodium sulphate, if they do not simultaneously have an alkalizing activity, such as sodium carbonate and sodium silicate. Suitable corrosion inhibitors include, more particularly, alkylphosphonic acids, of which octane phosphonic acid is particularly preferred. Further possible auxiliaries include dyes, perfume and solubilizing additives.

In use, the disinfectant is normally diluted with water.

It is preferably dissolved in water in a quantity of 1 to 10% by weight.

In a preferred embodiment, the disinfectant according to the invention is used for disinfecting surfaces and/or instruments.

It is also preferable to use the disinfectant according to the invention to destroy gram-positive bacteria and/or to destroy mycobacteria and/or to destroy viruses.

### EXAMPLES

### 1. Production of the Active Ingredient Solution

Three different powdered mixtures each consisting of 50% by weight sodium perborate monohydrate and 25% by weight TAED powder and

a) no surfactant

b) 2% by weight ABS (alkylbenzenesulfonate) and

c) 2% by weight Dehydol 980

were used as starting materials.

Corrosion inhibitors, complexing agents and further inorganic salts were used to make up 100% by weight.

The capacity of these different powder formulations 1a) to 1c) to dissolve in water was investigated without stirring or other movement in a first experiment.

For this purpose, 8 g of each of the powder formulations 1a) to 1c) were introduced into 100 ml of tap water respectively at room temperature.

It was found that formulations 1a) and 1b) required more than 1 hour to dissolve. In addition, a sediment also formed in the case of powder formulation 1b).

In the case of powder formulation 1c), on the other hand, the powder had dissolved within 1 hour. No sediment had formed either.

### Explanation

Dehydol 980 is an ether alcohol which is methyl-branched in the 2-position, of formula
R—O—(PO)₁₋₂-(EO)₆₋₈-H (I)
wherein the alkyl and alkenyl radicals R are made up as follows:

C₈=0 to 5% by weight;

C₉₋₁₀=75 to 90% by weight;

C₁₁₋₁₂=5 to 15% by weight;

C₁₃₋₁₄=4 to 10% by weight;

C₁₅₋₁₆=0 to 3% by weight.

### 2. Test for Effectiveness Against the Gram-Positive Bacterium Enterococcus hirae

Application solutions for microbiological investigations were prepared using powder formulations 1a) and 1c) by dissolving 12.5 g of each in 100 ml of tap water respectively.

These solutions were tested for the gram-positive bacterium Enterococcus hirae by the quantitative germ carrier test under dirty conditions in accordance with the new DGHM guideline (Status 1.3.2001). The following log reduction factors (triple determination in each case) were thus established:

| Contact time | 1c) | 1a) |
|---|---|---|
| 1 min | 3.06/2.23/3.1 | 0.53/0.61/0.48 |
| 5 min | 3.22/3.81/2.98 | 1.74/1.41/1.33 |
| 10 min | 6.8/6.8/6.8 | 3.72/3.46/3.8 |

The antimicrobial activity was a further advantage detected in the formulations according to the invention.

### 3. Test for Activity Against the Poliovirus

Application solutions for microbiological investigations were prepared using powder formulations 1a) and 1c) by dissolving 12.5 g of each in 100 ml of tap water respectively.

These solutions were tested against the poliovirus in the quantitative suspension test in accordance with the DVV guideline. The following average log reduction factors (triple determination in each case) were thus established:

| Contact time | 1c) | 1a) |
|---|---|---|
| 5 min | 1.5 | 1.5 |
| 10 min | 3.3 | 2.6 |

The virological test results thus reveal a further advantage.

1.-9. (canceled)

10. A method of disinfecting a surface in the medical field comprising applying an effective amount of a disinfectant use solution to the surface, wherein the disinfectant use solution comprises:
about 1 to about 10 wt. % of a powder diluted in water, said powder comprising:
(i) an uncoated peroxide;
(ii) an acylating agent; and
(iii) a nonionic surfactant selected from the group consisting of linear or 2-methyl-branched ether alcohols having the formula
R—O—(PO)₁₋₂-(EO)₆₋₈-H
wherein R is
C₈=0 to 5% by weight of the nonionic surfactant in the composition;
C₉₋₁₀=75 to 90% by weight of the nonionic surfactant in the composition;
C₁₁₋₁₂=5 to 15% by weight of the nonionic surfactant in the composition;
C₁₃₋₁₄=4 to 10% by weight of the nonionic surfactant in the composition;
and
C₁₅₋₁₆=0 to 3% by weight of the nonionic surfactant in the composition and the nonionic surfactant is free of alkoxylated alkyl phenol, wherein the nonionic surfactant is present in an amount effective to improve the dissolution rate of the powder composition in water.

11. The method of claim 10, wherein the surface is a medical instrument.

12. The method of claim 10, wherein the peroxide is selected from the group consisting of sodium perborate monohydrate, sodium perborate tetrahydrate, sodium percarbonate, and mixtures thereof.

13. The method of claim 10, wherein the acylating agent is selected from the group consisting of tetraacetyl glycoluril, tetraacetyl ethylenediamine, diacetyl hexahydrotriaone dione, and mixtures thereof.

14. The method of claim 10, wherein the powder comprises:
from about 10 to about 70 wt. % peroxide;
from about 10 to about 40 wt. % acylating agent; and
from about 0.1 to about 10 wt. % nonionic surfactant.

15. The method of claim 10, wherein the powder comprises:
from about 15 to about 60 wt. % peroxide;
from about 15 to about 30 wt. % acylating agent; and
from about 0.5 to about 5 wt. % nonionic surfactant.

16. The method of claim 10, wherein the powder further comprises a soluble inorganic salt.

17. The method of claim 10, wherein the powder further comprises additional auxiliary ingredients.

18. The method of claim 10, wherein the additional auxiliary ingredients are selected from the group consisting of alkalising agents, complexing agents, corrosion inhibitors, and surfactants.

19. A method of forming a disinfectant use solution for disinfecting a surface in the medical field comprising:
(a) providing a powder composition comprising:
(i) an uncoated peroxide;
(ii) an acylating agent; and
(iii) a nonionic surfactant selected from the group consisting of linear or 2-methyl-branched ether alcohols having the formula
R—O—(PO)₁₋₂-(EO)₆₋₈-H
wherein R is
C₈=0 to 5% by weight of the nonionic surfactant in the composition;
C₉₋₁₀=75 to 90% by weight of the nonionic surfactant in the composition;
C₁₁₋₁₂=5 to 15% by weight of the nonionic surfactant in the composition;
C₁₃₋₁₄=4 to 10% by weight of the nonionic surfactant in the composition;
and
C₁₅₋₁₆=0 to 3% by weight of the nonionic surfactant in the composition and the nonionic surfactant is free of alkoxylated alkyl phenol, wherein the nonionic surfactant is present in an amount effective to improve the dissolution rate of the powder composition in water; and
(b) mixing from about 1 to about 10 wt. % of the powder composition with water to form a disinfectant use solution.

20. The method of claim 19, wherein the surface is a medical instrument.

21. The method of claim 19, wherein the peroxide is selected from the group consisting of sodium perborate monohydrate, sodium perborate tetrahydrate, sodium percarbonate, and mixtures thereof.

22. The method of claim 19, wherein the acylating agent is selected from the group consisting of tetraacetyl glycoluril, tetraacetyl ethylenediamine, diacetyl hexahydrotriaone dione, and mixtures thereof.

23. The method of claim 19, wherein the powder comprises:
from about 10 to about 70 wt. % peroxide;
from about 10 to about 40 wt. % acylating agent; and
from about 0.1 to about 10 wt. % nonionic surfactant.

24. The method of claim 19, wherein the powder comprises:
from about 15 to about 60 wt. % peroxide;
from about 15 to about 30 wt. % acylating agent; and
from about 0.5 to about 5 wt. % nonionic surfactant.

25. The method of claim 10, wherein the powder further comprises a soluble inorganic salt.

26. The method of claim 10, wherein the powder further comprises additional auxiliary ingredients.

27. The method of claim 26, wherein the additional auxiliary ingredients are selected from the group consisting of alkalising agents, complexing agents, corrosion inhibitors, and surfactants.
